(19) United States
(12) Patent Application Publication
(10) Pub. No.: US 2013/0017247 A1
(43) Pub. Date: Jan. 17, 2013

(54) TREATMENT AND/OR PREVENTION OF ORAL ALLERGIC SYMPTOMS CAUSED BY ORAL CONTACT WITH FRUITS AND/OR VEGETABLES

(71) Applicants: Ziv Harish, Tenafly, NJ (US); Isaac Rubinstein, Haworth, NJ (US); Russ Weinzimmer, Milford, NH (US); Ehud Arbit, Englewood, NJ (US)

(72) Inventors: Ziv Harish, Tenafly, NJ (US); Isaac Rubinstein, Haworth, NJ (US); Russ Weinzimmer, Milford, NH (US); Ehud Arbit, Englewood, NJ (US)

(21) Appl. No.: 13/352,277

(22) Filed: Jan. 17, 2012

Related U.S. Application Data
(62) Division of application No. 12/981,726, filed on Dec. 30, 2010, now abandoned.

(51) Int. Cl. A61K 31/352; A61P 37/08; A61K 9/127

(52) U.S. Cl. 424/450; 514/456

(57) ABSTRACT

Treatment or prevention of oral allergy syndrome, or other allergy-related oral inflammatory processes using a topical preparation of at least one of a mast cell stabilizer and an antihistamine is disclosed. For example, the topical administration of Cromolyn Sodium prior to ingestion of raw fruits and raw vegetables may be used to prevent oral allergy symptoms. The topical administration can be performed by use of a solution, a powder, an aerosol, a tablet, a chewable tablet, a gel, a melting strip, a mouth rinse, a mouth swab, or a piece of candy, and may be combined with carrier molecules and/or mucoadhesive molecules to enhance efficacy.

### CROSS-REFERENCES TO RELATED APPLICATIONS

This application is a divisional of co-pending U.S. application Ser. No. 12/981,726 filed on Dec. 30, 2010.

### FIELD OF THE INVENTION

This invention relates to prevention and/or treatment of oral allergy, and preparations for topical prevention and/or topical treatment of oral allergy symptoms.

### BACKGROUND OF THE INVENTION

Allergic conditions result from release of mediators, including histamine, from sensitized mast cells upon exposure to an allergen.

Individuals with pollen allergy often experience oral itchiness, i.e., irritation of various surfaces within the mouth and throat, upon ingestion of raw fruits and/ or raw vegetables, often resulting in these individuals deciding to avoid eating these foods. The condition of oral itchiness, or other allergic symptom, such as irritation and/or swelling, is the result of local activation of mast cells within tissues of the mouth upon exposure to raw fruits and vegetables that cross-react with the pollens to which the individuals are allergic. As defined herein, “oral itchiness” shall also refer to any allergic symptom affecting the throat (pharynx) and/or the voice box (larynx), such a itchiness, irritation, and/or swelling thereof.

Currently, no approved medical treatment is available to alleviate this condition. Patients are advised to avoid raw fruits to which they are sensitive, but this deprives the patient of important nutrients that are found in fruits and vegetables. Avoidance also causes uncomfortable social problems. There are anecdotal reports of relief with allergy immunotherapy (allergy shots), and with use of antihistamines.

Mast cell stabilizers, such as Cromolyn Sodium, are commonly used in treatment of multiple allergic conditions, including asthma, rhinoconjunctivitis, eczema, and pruritus. Cromolyn Sodium is a salt of Cromoglicic acid, and is available in multiple forms: as a nasal spray (Rynacrom (UK), Nasalcrom, Prevalin (Netherlands)) to treat allergic rhinitis; in a nebulizer solution for aerosol administration to treat asthma; and as an inhaler (Intal) for preventive management of asthma. The maker of Intal™, King Pharmaceuticals, has discontinued manufacturing the inhaled form, cromolyn sodium inhalation aerosol, due to issues involving CFC-free propellant. As stocks are depleted, this inhaler preparation will no longer be available to patients.

Cromolyn Sodium is also available as eye drops (Opticrom™ and Optrex Allergy™ (UK), Crolom™) for allergic conjunctivitis; and also in an oral form (Gastrocrom™) to treat mastocytosis, dermatographic urticaria and ulcerative colitis. Another oral product, Intercron™ (sodium cromoglicate in distilled water, from Zambon France), is used for systemic treatment of food allergies. However, the physicochemical properties of cromolyn impede its absorption across epithelial lining such as through the oral mucosa and contribute to its poor absorption from the gastrointestinal tract. No more than 1% of a dose administered orally is absorbed by humans, the remainder being excreted in the feces. Very little absorption of Cromolyn Sodium was seen after administration of 500 mg by mouth to each of 12 volunteers. From 0.28 to 0.50% of the administered dose was recovered in the first 24 hours of urinary excretion in 3 subjects. The mean urinary excretion of an orally administered dose over 24 hours in the remaining 9 subjects was 0.45%.

Each 5 mL ampule of Gastrocrom™ contains 100 mg Cromolyn Sodium, USP, in purified water. Cromolyn Sodium, USP, is a hygroscopic, white powder having little odor. It may leave a slightly bitter aftertaste. Gastrocrom™ Oral Concentrate is clear, colorless, and sterile. It is intended for “oral” use, i.e., to be swallowed and reach superficially situated mast cells along the gastrointestinal tract, or to be absorbed systemically via the gastrointestinal tract, just as pills, capsules, and tablets are to be swallowed and absorbed systemically.

In particular, users of Gastrocrom™ are instructed to: Squeeze liquid contents of an ampule containing a single dose of Gastrocrom™ into a glass of water. Stir the resulting solution. Drink all of the solution immediately.

Also, Cromolyn Sodium is commonly administered via a power-driven nebulizer, under the brand names: Intal™ and Intal Inhaler™. The nebulizer must have a suitable face mask or mouthpiece. Nebulizers make a liquid into an aerosol that one breathes in through one's mouth, or through one's mouth and nose, into the lungs. However, one is also commonly advised: “Do not take this medicine as a solution by mouth”. One is also advised “If you get a bitter or unpleasant taste in your mouth, gargle or rinse your mouth after you use this medicine.” Patients are also advised: “To relieve dry mouth or throat irritation caused by cromolyn inhalation, rinse your mouth with water, chew gum, or suck sugarless hard candy after each treatment.”

Patients are advised to: “Use cromolyn sodium inhalation on a regular basis for best results. It may be several weeks before you see the effects.” Patients are further advised: “It is very important that you use your cromolyn sodium inhaler properly, so that the medicine gets into your lungs. Your doctor may want you to use a spacer with your inhaler.” Cromolyn oral inhalation will no longer be available in the U.S. after Dec. 31, 2010.

Because of their convenience (and perceived safety), leukotriene receptor antagonists have largely replaced Cromoglicic acid and its salt Cromalyn Sodium, as the non-corticosteroid treatment of choice. Stated disadvantages include: “Cromoglicic acid requires administration four times daily, and does not provide additive benefit in combination with inhaled corticosteroids.”

It is commonly accepted that Cromolyn Sodium is poorly absorbed topically through mucosal barriers, and is consequently not absorbed well into the oral mucosa. Further, it is also commonly believed that the physicochemical properties including the polarity of the Cromolyn Sodium molecule may also interfere with its absorption and local activity.

### SUMMARY OF THE INVENTION

One general aspect of the invention is a method for the treatment of oral allergic symptoms due to oral contact with a food allergen. The method includes topically administering an effective amount of at least one of: a mast cell stabilizer; and an antihistamine.

In a preferred embodiment, the mast cell stabilizer also has anti-histamine properties.

In a preferred embodiment, the mast cell stabilizer is mixed with the antihistamine.

In a preferred embodiment, at least one of the mast cell stabilizer and the antihistamine is mixed with a viscous carrier.

In a preferred embodiment, at least one of the mast cell stabilizer and the antihistamine is mixed with a mucoadhesive material so as to extend contact time with the oral mucosa.

In a preferred embodiment, the mucoadhesive material includes at least one of: a cyclodextrin, chytosan, alginate, glycerine, propylene glycol.

In a preferred embodiment, the mast cell stabilizer is Cromolyn Sodium.

In a preferred embodiment, the mast cell stabilizer is mixed with a carrier molecule so as to enhance local and/or systemic absorption.

In a preferred embodiment, the carrier molecule is sodium N-(8[2-hydroxybenzoyl]amino) caprylate, (SNAC).

In a preferred embodiment, an effective amount of at least one of a mast cell stabilizer and an antihistamine is included in at least one of: a pump spray, an aerosol spray, a nebulizer, a liposomal suspension, a mouth wash, an mouth rinse, an oral swab, a gel, a sublingual, a lollypop, a piece of candy, a melting tablet.

In another preferred embodiment, the mast cell stabilizer is mixed with an absorption enhancer, such as ethylenediaminetetraacetic acid (EDTA), Cyclodextrine, a surfactant, or a bile acid, so as to enhance local and/or systemic absorption.

In another preferred embodiment, the absortion enhancer is in the form of a carrier molecule such as sodium N-(8[2-hydroxybenzoyl]amino) caprylate, (SNAC) or N-(5-chlorosalicyloyl)-8-aminocaprylic acid (5-CNAC).

Another general aspect of the invention is the use of a mast cell stabilizer in the manufacture of a medicament for the treatment of oral allergic symptoms.

Another general aspect of the invention is the use of a mast cell stabilizer in the manufacture of a preparation for the prevention of oral allergic symptoms.

Another general aspect of the invention is a method of treatment of oral allergy to a raw fruit and/or a raw vegetable, where the method includes: topical administration of a therapeutically effective amount of at least a mast cell stabilizer to a patient in need of such treatment.

Another general aspect of the invention is a pharmaceutical formulation for use in the prevention of oral allergic symptoms of a person, the pharmaceutical preparation including: an effective amount of at least one of: a mast cell stabilizer and an antihistamine; and a delivery medium capable of prolonging contact time between the mast cell stabilizer and/or the antihistamine, and at least a portion of oral mucosa of the person.

In a preferred embodiment, the mast cell stabilizer is selected from: Cromolyn Sodium, Cromoglicate (cromolyn), Nedocromil, β2 adrenergic agonists, Pemirolast potassium, Nedocromil sodium, Lodoxamide tromethamine, Bepotastine besilate, Epinastein HCl, Azelastine HCl, Olopatadine HCl, Ketotifen fumarate.

In a preferred embodiment, the antihistamine is selected from: first generation antihistamines, and second generation antihistamines.

In a preferred embodiment, the oral allergic symptoms of the person are due to the consumption of at least one of: a piece of raw fruit, a piece of raw vegetable, a raw nut, fish, shellfish, dairy, baked goods, eggs.

In a preferred embodiment, the delivery medium is selected from: a thickening agent, a viscous agent, a wetting agent, an adhesive agent.

In a preferred embodiment, the adhesive agent is selected from: cyclodextrins, chytosan, alginate, honey, syrup, glycerine, propylene glycol.

In a preferred embodiment, the pharmaceutical formulation is configured to be delivered to oral mucosa of the person as one of: a syrup, a gel, a spray, a mouthwash, a mouth rinse, a swab, a nebulization, a capsule, a sublingual tablet, a dissolving tablet, a candy, a melting strip, a lollipop, a liposomal suspension, quick-dissolving strips, rapid disintegrating tablets, oral dissolving tablets.

In a preferred embodiment, the pharmaceutical formulation further includes: an absorption enhancing element, such as SNAC.

In a preferred embodiment, the delivery medium includes a flavoring compound, or a taste-masking compound.

Another general aspect of the invention is a method for the prevention of oral allergic symptoms due to oral contact with a food allergen, the method including: topically administering, substantially before consuming the food allergen, an effective amount of at least one of: a mast cell stabilizer; and an antihistamine.

In a preferred embodiment, the mast cell stabilizer also has anti-histamine properties.

Another general aspect of the invention is a pharmaceutical composition for the prevention of oral allergy symptoms via topical application, the pharmaceutical composition including: at least one of a mast cell stabilizer and an antihistamine; and a carrier for prolonging contact of at least one of the mast cell stabilizer and the antihistamine with oral mucosa expected to exhibit the oral allergy symptoms.

Another general aspect of the invention is the use of a mast cell stabilizer for the manufacture of a pharmaceutical composition for the topical treatment of oral allergy.

Another general aspect of the invention is the use of a mast cell stabilizer for the manufacture of a pharmaceutical composition for the topical prevention of oral allergy.

Another general aspect of the invention is the use of an antihistamine for the manufacture of a pharmaceutical composition for the topical treatment of oral allergy.

Another general aspect of the invention is the use of an antihistamine for the manufacture of a pharmaceutical composition for the topical prevention of oral allergy.

### DETAILED DESCRIPTION

Activation of mucosal mast cells in the oral cavity upon ingestion of raw fruits and/or raw vegetables may result in oral itchiness, that often precludes affected individuals from consuming raw fruits and vegetables. This may significantly adversely affect the allergic individual's quality of life, due to both personal and social implications, and it may compromise self-esteem especially in the pediatric age group, and may further raise nutritional concerns.

According to the invention, blocking the release of histamine and other mediators from the mast cells by mast cell stabilizers, such as Cromolyn Sodium, may enhance the ability of affected individuals to consume raw fruits and raw vegetables that otherwise would trigger an allergic reaction.

The proposed invention consists of introduction of a mast cell stabilizer, such as Cromolyn Sodium, prior to ingestion of fruits and vegetables, either through swallowing, e.g. as tablets, as capsules, or as a liquid or topical application, such as in the form of melting tabs, aerosol, oral wash, swabs, candy, or lollypop. Topical application of the mast cell stabilizer before consuming a food allergen, such as raw fruit or raw vegetable, will PREVENT or usefully minimize the uncomfortable oral allergic reaction, such as itching, swelling, etc.

Antihistamine (either first generation, or second generation) can be added to the topical formulation of the mast cell stabilizer, or it can replace it.

For example, to overcome its poor mucous-membrane absorption, Cromolyn Sodium may be mixed in a viscous solution as well as with mucoadhesive materials, such as cyclodextrins, chytosan, alginate, glycerine, propylene glycol so as to extend contact time between Cromolyn Sodium and the oral mucosa and increase the residence time of cromolyn on top of the mucosa by forming a coating over this membrane. Alternatively, or in combination with the foregoing, or Cromolyn Sodium may be mixed with drug absorption enhancers (e.g. EDTA, or Cyclodextrine or a surfactant—e.g polyoxyethelene 9-lauryl esther, Tween 60, poly acrylic acid, or bile acids—sodium cholate, sodium glycocholate, lauryl sulfate) or with carrier molecule which increase drug absorption, such as the carrier molecule, sodium N-(8[2-hydroxybenzoyl]amino) caprylate, referred to as “SNAC”, to enhance its local and systemic absorption, and thereby maximize its effect.

Take the preparation of the invention five to fifteen minutes before eating a raw fruit that is expected to cause an oral allergy reaction. Take a test amount of the fruit to verify that no symptoms are experienced, i.e., that the oral allergic reaction has in fact been prevented.

An example of a safe and effective formulation is to combine 100 mg of Cromolyn Sodium, in 5 cc water (e.g.Gastrocrom™) with 100 mg of glycerin to create an oral solution. The glycerine helps the patient keep it in the mouth. Alternatively, instead of glycerine, glycerin alginate can be used as a 1% to 10% solution in water (e.g. 1 mg alginate/100 ml water). Glycerol can also be used, usually in the 5-20% concentration range, and propylene glycol 1-10% can be used also. All are GRAS.

A flavoring and/or sweetener can be added to make the preparation more palatable. Or, the mast cell stabilizer can be formulated as part of a mouth rinse, having generally the same surfactant properties as Anti-Plaque mouth rinse to help the preparation dwell longer in mouth.

Cromolyn Sodium must be taken into and topically applied to the oral mucosa so as to create a four-hour effectiveness window. Nevertheless, the preparation of the invention can be effective even when taken just a few minutes before eating fruit.

Cromolyn Sodium is poorly absorbed through mucosal barriers unless it is attached to a carrier molecule. Topical application of Cromolyn Sodium when attached to a carrier molecule is enhanced in effectiveness.

However, the polarity of the Cromolyn Sodium molecule may also interfere with its local and/or topical activity, and thus to maximize its effect, it may be embedded in a viscous solution, or it can be mixed with mucoadhesive. Another benefit of the preparation of the invention is that swallowing this preparation is NOT needed, thereby reducing the amount of drug to which a patient would be exposed.

When using an anti-histamine either alone or in combination with a mast cell stabilizer, the anti-histamine should work fast, and be less sedating. The mast cell stabilizer selected should be the most effective mast cell inhibitor, i.e., a mast cell inhibitor of known efficacy in a variety of allergic conditions, with an excellent safety record, with minimal unpleasant taste. Also possibly combine with a taste masker, such as a flavor and/or a sweetener.

Suiteable Mast cells stabilizers include:
Alamast™; Vistakon/Pemirolast potassium.
Alocril™; Allergan/Nedocromil sodium.
Alomide™; Alcon/Lodoxamide tromethamine.
Bepreve™; Ista/Bepotastine besilate.
Crolom™; Bausch & Lomb/Cromolyn sodium.
Elestat™; Inspire/Epinastein HCl.
Optivar™; Meda/Azelastine HCl.
Pataday™; Alcon/Olopatadine HCl.
Patanol™; Alcon/Olopatadine HCl.
Zaditor™; Novartis Ophthalmics OTC/Ketotifen (as fumarate).

The medications that are listed below are listed as Mast cell stabilizers for the eye.

Those that are also antihistamines are marked: “also an antihistamine.” These all can be used as mast cell stabilizers in preparations of the invention:
Zyrtec Itchy eye™; McNeil/ketotifen fumarate also an antihistamine.
Elestat™; Allergan/epinastine HCl also an antihistamine.
Claritin Eye™; Merck/Ketotifen also an antihistamine.
Alaway™; Bausch & Lomb/ketotifen fumarate also an antihistamine.
Alamast™; Vistakon/Pemirolast potassium.
Alocril™; Allergan/Nedocromil sodium.
Alomide™; Alcon/Lodoxamide tromethamine.
Bepreve™; Ista/Bepotastine besilate. also an antihistamine.
Crolom™; Bausch & Lomb/Cromolyn sodium.
Elestat™; Inspire/Epinastein HCl.
Optivar™; Meda/Azelastine HCl. also an antihistamine.
Pataday™; Alcon/Olopatadine HCl. also an antihistamine.
Patanol™; Alcon/Olopatadine HCl. also an antihistamine.
Zaditor™; Novartis Ophthalmics OTC/Ketotifen (as fumarate). also an antihistamine.

Below are medications that are listed only as anti histamines:
Emadine™; Alcon/Emedastine difumarate
Naphcon-A;Alcon/Naphazoline HCl.
Visin-A™; McNeil/Naphazoline HCl

The preparation can be applied topically as a mouthwash that includes glycerin. It must stay in the mouth for a short while (15 to 90 seconds), long enough to be in contact with the oral mucosa and form a temporary coating on its surface. It will begin its effects within a short time after contact with the mucosa, about one to fifteen minutes.

The oral preparation can be used ad-hoc before ingestion of an allergenic food, or preventively, one to four times a day.

The preparation of the invention can also be applied topically as a mouth spray, or by a liposomal emulsion. A liposomal emulsion formulation easily enters the oral mucosa, thereby providing enhanced effectiveness. Much like human cells, liposomes encapsulate the active ingredients of the preparation within a lipid bilayer, thus enabling them to penetrate readily into the mucosa of the mouth.

Oral allergy can also occur upon consumption of raw nuts. The pharynx and larynx can be involved with swelling and or itching. The invention can also be used for prevention and/or treatment of minor local allergic reaction within the mouth to any food, such as cake that includes eggs and/or milk and/or soy and/or fish and/or shell-fish. Oral allergy predominantly occurs when the fruits and vegetables are raw.

Topical antihistamines such as diphenhydramine (Benadryl) work well as an antihistamine incorporated into the preparation of the invention.
Local anesthetics for topical use such as benzocaine (currently used as a topical cream Lanacaine™ or Pramocaine™), can also advantageously be included in the preparation of the invention to further decrease discomfort due to oral allergy symptoms.

Counter-irritants, such as mint oil, menthol, or camphor—can be advantageously included in preparations of the invention, alone, or in combination with other active ingredients therein, such as Cromolyn Sodium.

Flavoring compounds and/or taste-masking compounds can also be advantageously included in the preparation of the invention. Even a preparation of Cromolyn Sodium, water, and a flavoring compound (e.g., Strawberry, Cherry, Lemon, Mint) can be used as an effective mouth rinse or mouth wash for preventing and/or treating oral allergy symptoms. Also, a preparation of Cromolyn Sodium and a flavor masking compound to mask any unpleasant taste of the Cromolyn Sodium can be used as an effective mouth rinse or mouth wash for preventing and/or treating oral allergy symptoms.

Cromolyn Sodium has been approved by the U.S. Food & Drug Administration (FDA) for sale by prescription, and after decades of use, has an excellent safety record. Cromolyn Sodium in nasal form (NasalCrom™) is presently sold OTC in the USA. Cromolyn Sodium has been used for decades in treatment of multiple allergic conditions including asthma, but not yet for the prevention and/or treatment of oral allergy symptoms, including Oral Allergy Syndrome. Oral allergy symptoms include the oral itchiness associated with ingestion of raw fruits, such as raw apples, and raw vegetables, such as raw carrots.

Side effects of Cromolyn Sodium are considered very uncommon and when swallowed, may include headache; GI upset; itchiness; irritability; rash; and muscle pain. Since the preparation of the invention provides oral exposure without swallowing the drug, side effects are expected to be even less common. Cromolyn Sodium is a generic drug name for a mast cell stabilizer, and is sold under trade names that include: Gastrocrom™; Opticrom™; Crolom™; Nasalcrom™ and Intal™.

Other modifications and implementations will occur to those skilled in the art without departing from the spirit and the scope of the invention as claimed. Accordingly, the above description is not intended to limit the invention, except as indicated in the following claims.

What is claimed is:

1. A method for the treatment of oral allergic symptoms due to oral contact with a food allergen, the method comprising:
topically administering to the oral mucosa of an individual an effective amount of at least one of:
a mast cell stabilizer; and
an antihistamine.

2. The method of claim 1, wherein the mast cell stabilizer also has anti-histamine properties.

3. The method of claim 1, wherein the mast cell stabilizer is mixed with the antihistamine.

4. The method of claim 1, wherein at least one of the mast cell stabilizer and the antihistamine is mixed with a viscous carrier.

5. The method of claim 1, wherein at least one of the mast cell stabilizer and the antihistamine is mixed with a mucoadhesive material to extend contact time with the oral mucosa.

6. The method of claim 5, wherein the mucoadhesive material includes at least one of:
a cyclodextrin, chytosan, alginate, glycerine, propylene glycol.

7. The method of claim 1, wherein the mast cell stabilizer is Cromolyn Sodium.

8. The method of claim 1, wherein the mast cell stabilizer is mixed with a carrier molecule so as to enhance local and/or systemic absorption.

9. The method of claim 8, wherein the carrier molecule is sodium N-(8[2-hydroxybenzoyl]amino) caprylate, (SNAC).

10. The method of claim 1, wherein the effective amount of at least one of a mast cell stabilizer and an antihistamine is included in at least one of:
a pump spray, an aerosol spray, a nebulizer, a liposomal suspension, a mouth wash, an mouth rinse, an oral swab, a gel, a sublingual, a lollypop, a piece of candy, a melting tablet.

11. A method of treatment of oral allergy symptoms due to oral contact with a food allergen, the method comprising:
topical administration of a therapeutically effective amount of cromolyn sodium to a patient in need of such treatment.

12. The method of claim 9, wherein the therapeutically effective amount of cromolyn sodium is mixed with a mucoadhesive material to extend contact time with the oral mucosa.

13. The method of claim 12, wherein the mucoadhesive material includes at least one of:
a cyclodextrin, chytosan, alginate, glycerine, propylene glycol.

14. The method of claim 11, wherein the therapeutically effective amount of cromolyn sodium is included in at least one of:
a pump spray, an aerosol spray, a nebulizer, a liposomal suspension, a mouth wash, an mouth rinse, an oral swab, a gel, a sublingual, a lollypop, a piece of candy, a melting tablet.

15. The method of claim 11, wherein the therapeutically effective amount of cromolyn sodium is mixed with an absorption enhancer, such as ethylenediaminetetraacetic acid (EDTA), Cyclodextrine, a surfactant, or a bile acid, to enhance local and/or systemic absorption.

16. The method of claim 11 wherein the therapeutically effective amount of cromolyn sodium is mixed with an absorption enhancer in the form of a carrier molecule such as sodium N-(8[2-hydroxybenzoyl]amino) caprylate, (SNAC) or N-(5-chlorosalicyloyl)-8-aminocaprylic acid (5-CNAC).

17. A method for the prevention of oral allergic symptoms due to oral contact with a food allergen, the method comprising:
topically administering, substantially before consuming the food allergen, an effective amount of cromolyn sodium to the oral mucosa of an individual susceptible to such symptoms.

18. The method of claim 17, wherein the effective amount of cromolyn sodium is mixed with a viscous carrier.

19. The method of claim 17, wherein the effective amount of cromolyn sodium is mixed with a mucoadhesive material so as to extend contact time with the oral mucosa.
